(19) United States
(12) Patent Application Publication
(10) Pub. No.: US 2017/0136203 A1
(43) Pub. Date: May 18, 2017

(54) METHOD AND SYSTEM FOR INCREASING BROWN ADIPOSE TISSUE ACTIVITY DURING SLEEP

(71) Applicant: DOUG J. SWAIN, Griswold, IA (US)

(72) Inventor: DOUG J. SWAIN, Griswold, IA (US)

(21) Appl. No.: 14/941,797

(22) Filed: Nov. 16, 2015

(51) Int. Cl. A61M 16/10; A61M 16/08; A61M 16/16; A61F 5/00; A61M 16/06

(52) U.S. Cl. CPC A61M 16/108; A61F 5/0003; A61M 16/06; A61M 16/16; A61M 16/0808; A61M 16/0875; A61M 2230/50; A61M 2205/50

(57) ABSTRACT

A method and system is provided for increasing brown adipose tissue deposits and activity by breathing air from a CPAP machine having a chiller which cools the air to less than 55° F. before discharge from the CPAP mask to the person's lungs while sleeping. The cool air increases brown adipose tissue development and activity, thereby increasing caloric expenditures, so as to lead to weight loss and other health benefits.

### BACKGROUND OF THE INVENTION

Obesity and diabetes are contemporary pandemics which continue to grow in numbers and the breadth of people affected. Obesity develops over time when energy intake exceeds energy expenditure. Most commonly, obesity is the result of an imbalance between the ingestion of energy-dense foods and decreased physical activity. The excess calories from too much food and not enough exercise is normally stored in white adipose tissue.

The pathological accumulation of excess, dysfunctional white adipose tissue that characterizes obesity is a major risk factor for many other diseases, including Type2 diabetes, cardiovascular disease, hypertension, stroke, arthritis, and various types of cancer. A basic concept is that weight gain is caused by a fundamental energy imbalance, when energy intake from food chronically exceeds energy expended by physical activity and metabolical processes in humans. Therefore, the common therapy for weight loss involves a decrease in food intake and/or an increase in energy expenditure.

Brown adipose tissue is present to a much greater extent in newborn humans, and defends core body temperature by stimulating heat production in the body. However, the presence of BAT in newborn humans regresses shortly after birth. While small deposits of BAT in adult humans has been known, the general belief was that human BAT had a negligible impact on energy homeostasis. Only in the last decade has the importance and function of brown adipose tissue in adult humans begun to be understood.

Most adult human fat consists of white adipose tissue, which is energy-storing. In recent years, research has discovered the ability to induce brown adipose tissue (BAT) in adult humans. The brown adipose tissue has 400 times more thermogenic capacity to burn energy to make heat than any other tissue. This functional benefit of brown adipose tissue, also known as brown fat, creates potential therapeutic benefits for inducing weight loss and preventing/treating associated diseases, and helping to control type II diabetes through decreased insulin resistance.

There appears to be an inverse correlation between brown adipose tissue (BAT) activity/abundance and ambient air temperature, such that adult human BAT has increased activity/abundance from cold stimulation. Increasing BAT activity to dissipate energy, and thereby burn calories, has led medical research to target BAT for reduction or prevention of obesity and its associated diseases. Relatively recent research suggests that long term exposure to cold environments can stimulate recruitment of BAT in adult humans, thereby increasing energy metabolism. For example, in one 4 month study, participants slept in a room wherein the room temperature was lowered the 2^{nd} month from 24° C. (75° F.) to 19° C. (66° F.). The results showed an increase in the amount and activity of brown fat by approximately 30%-40% and increased whole body tissue consumption of glucose during the month of exposure to the colder room air. After the second month, another month of sleeping in warmer room air at 27° C. (80° F.) showed a decrease in the amount of brown fat in the participants, demonstrating that brown fat requires routine stimulation to persist and is not permanent once obtained. However, exposure of humans to such cooler than normal air temperatures for extended periods would require increased (clothing or blankets) for added warmth and comfort. Absent such additional insulation, the body will naturally and eventually begin to shiver if a person is awake or awaken the person if core body temperature drops to severe. Cold stimulation also stimulates the satiety center in the brain inducing a hunger feeling when awake. Many people, when hungry, will eat, which is the antithesis of weight loss or control. Also, if a spouse or partner sleeps in the same bed in the cold air room, the discomfort may make sleeping more difficult for the second person. Studies suggest that people do not have a shivering reflex during REM sleep.

Therefore, a primary objective of the present invention is the provision of a method and system for delivering cold air to a person for breathing, so as to increase the quantity and activity of brown adipose tissue in a reasonably comfortable fashion to the patient.

Another objective of the present invention is the provision of a method of delivering cold air to a person during sleep, without changing the ambient air temperature in the sleeping room.

A further objective of the present invention is the provision of a system for reducing breathing air temperature during sleep.

Still another objective of the present invention is the provision of a method for increasing energy expenditure by the human body.

Yet another objective of the present invention is the provision of a method and system for stepping down breathing air temperature after a person enters initial REM sleep and increasing the breathing air temperature before the person exits final REM sleep.

Another objective of the present invention is the provision of a CPAP machine having an air chiller to provide cool air to a person wearing the CPAP mask.

Still another objective of the present invention is the provision of a method of increasing BAT deposits and activity which is safe and efficacious.

Another objective of the present invention is the provision of a method and system to reduce insulin resistance.

Yet another objective of the present invention is the provision of a method to deliver cold therapy to the patient with minimal condensation/rainout in the delivery tubing.

Still another objective of the present invention is the provision of a method to inherently produce distilled water product for the humidification process.

These and other objectives become apparent from the following description of the invention.

### SUMMARY OF THE INVENTION

Increasing brown adipose tissue deposits and activity is achieved by providing air through a CPAP mask at a temperature no more than 55° F. for breathing while the person is in REM or near REM sleep. The modified CPAP machine is provided with an air chiller to reduce ambient air temperature to a desired range. A humidifier can also be provided for humidifying the cool air prior to breathing. A microprocessor or CPU is programmed so as to step down the air temperature so that the coolest temperature is achieved after the person enters REM sleep, and steps up the air temperature before the person exits REM sleep. As the person cycles into and out of initial and final REM sleep stages, the air temperature discharged from the CPAP mask for breathing also cycles down and back up. The ambient temperature of the air in the sleeping room is not affected by the CPAP air chiller system or method. The person's core body temperature is maintained above hypothermia and the inhalant air is warmed if hypothermia (94 degrees F. or less) is induced.

### BRIEF DESCRIPTION OF THE DRAWINGS

FIG. 1 is a schematic view showing the system for delivering cold air to a sleeping person, according to the present invention.

FIG. 2 is a chart showing an example of a temperature/sleep regimen for increasing brown adipose tissue activity, according to the present invention.

FIG. 3 is a table showing typical daily caloric expenditures for a person using a conventional CPAP machine in a convention manner.

FIG. 4 is a table showing estimated daily caloric expenditures for a person using the modified CPAP machine in accordance with the present invention.

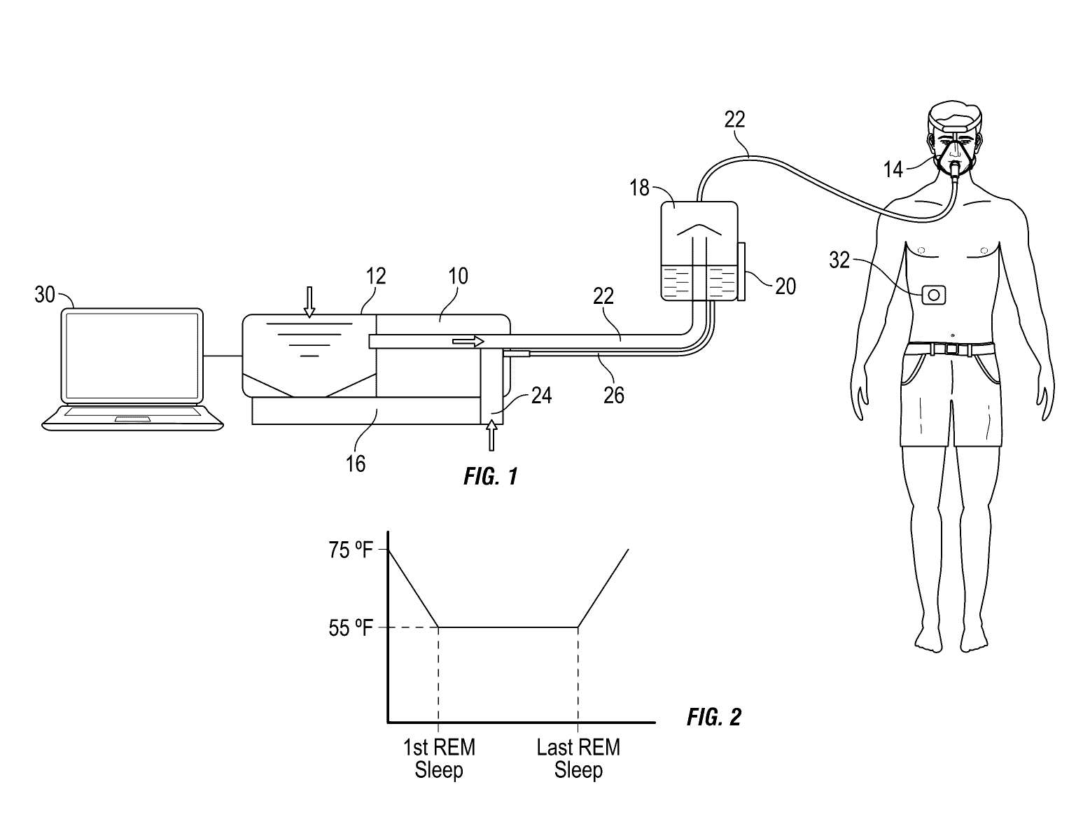

### DETAILED DESCRIPTION OF THE INVENTION

The method of increasing brown adipose tissue activity during sleep, according to the present invention, utilizes a modified CPAP machine 10. The machine 10 is modified to include an air chiller 12 which cools the temperature of the air before the air is sent to the CPAP mask 14 for breathing by a person. The chiller 12 can take any convenient form, such as a heat exchanger using air to air, fluid to air, or a conventional refrigeration process. More examples include chilling the air with liquid nitrogen or dry ice.

The machine 10 may also include a cavity or sump 16 to collect condensate generated by the chiller 12. The system preferably includes a humidifier 18 between the CPAP machine 10 and the mask 14 to add a comfortable level of humidity to the cool air prior to breathing. A heating element 20 may be included with the humidifier 18 to facilitate humidification of the air. As seen in FIG. 1, an air line 22 extends from the CPAP machine 10 to the humidifier 18, and then to the mask 14. A pump 24 on the machine 10 supplies distilled water from the sump 16 to the humidifier 18 via a fluid line 26.

Humidification chamber 18 is preferably positioned as close to the mask 14 as possible, because the air will acquire some heat gain in the delivery tubing and change relative humidity/water holding capacity prior to humidification. Also, conventional CPAP machines use hot air humidification, which requires heated tubing to prevent condensation in the delivery tubing, known as rain out. By minimizing the distance between the humidifier 18 and the mask 14, rainout is minimized through a decreased condensational surface area in the delivery tubing post humidification. As the cool air moves through the air line 22 from the chiller 12 to the humidifier 18, the temperature of the air will inherently increase due to the thermal heat gain through the tube 22 due to the higher room air temperature (68°-73° F.). To minimize heat gain, the air line 22 may be insulated, such that the temperature increases no more than ten degrees between the chiller 12 and the humidifier 18.

Relatively low humidity inhalant air to the mask 14 will burn more calories due to increased moisture vaporization from the mucus membranes in the lungs, airways, and sinus. The lower the air humidity from the mask 14, the more capacity the air has to carry vaporized water from the mucus membranes, thus allowing for more caloric expenditure for the person's body. However, if the air humidity is too low, the person may have nose, throat and sinus pain or irritation. Also, breathing extremely low humidity air (<50% relative humidity) decreases the ability of the mucociliary elevator in the trachea to rid dust and germs from entering the lower respiratory tract. Therefore, there is a need to balance the benefits and detriments of low humidity inhalant air. A preferred relative humidity range is 50%-60%, which should minimize risk of discomfort or sinusitis and respiratory tract infections.

Preferably, humidifier 18 should be as close as possible to the CPAP mask 14 so as to minimize or eliminate rain out wherein condensation forms in the air line 22 between the humidifier 18 and the mask 14. Positioning the humidifier 18 closer to the mask 14 also provides more consistent deliverable end-product humidification. Preferably, the humidification range is 50%-80%.

In the method of the present invention, the air is cooled by the chiller 12 to a level such that the temperature of the air provided by the mask 14 to the patient is preferably 40°-55° F. For example, the machine 10 can cool the air to approximately 34° or 35°, with the air temperature increasing approximately 20° in the delivery tubing and humidifier before breathing via the mask 14. Thus, the net difference between the ambient air in the sleeping room and the breathing air is approximately 20° F.

In the preferred embodiment, a computer or microprocessor or central processing unit 30 is provided with a software program to step down and step up the temperature of the air provided through the mask 14. For example, when the person first puts on the mask 14, the air being breathed is approximately the same temperature as the ambient room air temperature. After the person enters a first REM sleep cycle, the system steps down the air temperature to the 40°-55° F. range. Then, before the person exits their last REM sleep cycle, the system steps up the air temperature to be substantially equal to the ambient room temperature before the person awakens. Thus, the person does not sense the coolness of the air entering their upper respiratory tract.

If desired, a probe or sensor 32 can be attached to the person in any convenient manner so as to monitor the person's core body temperature, which should not fall below 94.1° F., so as to avoid hypothermia. The sensor 32 generates a signal which is transmitted to the CPU 30, which in turn regulates the chiller 12 so as to maintain the person's core body temperature above 94.1° F.

By inhaling the cool air for a substantial period of sleep, BAT activity/abundance is increased as to generate heat and to decrease insulin resistance. The person's lungs function as a physiologic heat exchanger. The cold breathing air thus causes the body to burn calories as the person sleeps, leading to weight loss and reduced health risks for diabetes and other obesity related diseases. Also, since the minimum temperature of the cold air is provided during the REM sleep cycles, a shiver response or reflex is avoided so as to avoid waking a sleeping partner in the same bed and avoid shivering discomfort of the patient. The method and system of the present invention does not require adjustment of the room air temperature.

In addition to burning calories, brown fat also decreases cell resistance to insulin. Increased brown fat percentages tend to lower average blood glucose levels. The cool air breathing system of the present invention induces brown fat deposition within the body and thereby lowers blood sugar levels so as to avoid type 2 diabetes, while the weight loss benefit is achieved due to the caloric expenditure of the highly metabolic brown fat and the thermal loss of calories through the relatively long term cold exposure of inhaled air.

It is understood that the software for the CPU 30 can be programmed to accommodate various sleep patterns and habits, including the time they go to sleep and wake up.

As an alternative to the core body temperature sensor 32, or in addition thereto, the present invention can use thermal imagery. For example, as blood shunts away from the periphery of the person's body (i.e. their arms and legs), thermal imagery data can be provided to the CPU 30 to indirectly correlate core body temperatures. Also, the thermal imagery 15 can indirectly measure brown fat deposition, since the brown fat cells put off more heat especially in the supraclavicular areas. Also, radio frequency technology can be utilized, such as a temperature microchip implanted in the person so as to generate a radio signal corresponding to the core body temperature for wireless transmission to the CPU.

When the person first puts on the CPAP mask 14 and turns on the CPAP machine 20, the chiller 12 is not activated, such that the air initially provided to the person is approximately room temperature at 68°-73 ° F., or higher. The humidifier 18 may be activated during this initial stage. Then, as the person enters the first REM sleep cycle, the chiller 12 is activated to cool the air temperature from the CPAP machine 10 to achieve an air breathing temperature of approximately 40°-55 ° F. The probe 32 monitors the person's core body temperature, and transmits a signal corresponding to the core body temperature to the CPU 30 so as to avoid hypothermia. The CPU 30 is programmed to control the chiller 12 and thereby raise the air breathing temperature back to ambient before the person awakes from their last REM sleep cycle.

In some instances, the CPU 30 may also control the chiller 12 so as to periodically increase and decrease the air temperature corresponding to the sequential REM sleep cycles which normally occur during a period of sleep. For example, in a preferred embodiment the CPU will gain data each night about the respiratory rate and tidal volumes. During transition from normal wake through non-REM sleep to REM sleep, tidal volumes will decrease (p value<0.05) and respiratory rate will increase. Range (7.66+/−0.34 L/min awake and 6.46+/−0.29 L/min Rem.) The breaths will be faster and shallower. The machine will initiate the cooling cycle as soon as tidal volumes fall and approach the 6.5 L/min threshold for set period of few minutes. The user will program his/her approximate time they sleep in hours. Because there is some variability in tidal volumes from REM to non-REM sleep as user goes in and out through the night; the machine will stay on the low air temp until 1-2 hours prior to wake period as programmed by the user. As soon as tidal volumes increase after this time, the warming cycle will be initiated. There will also be an override short cycle button so if bathroom breaks are uncomfortable while going back to sleep, a short cycle of warm air can be initiated. The machine will then function as if sleep was continuous and await wake time and sense increased tidal volume as normal prior to starting warm cycle.

Thus, by breathing relatively cold air during sleep, the person's body generates brown adipose tissue and increases the heat energy generated by the BAT during sleep and throughout the day during environmental cold exposure, thereby inducing weight loss, without long exposure to cold air while awake and without adjusting ambient air temperature in a room. The results of this increased metabolism and caloric burn can be coupled with reduced food intake and increased exercise to maximize the weight loss results and benefits.

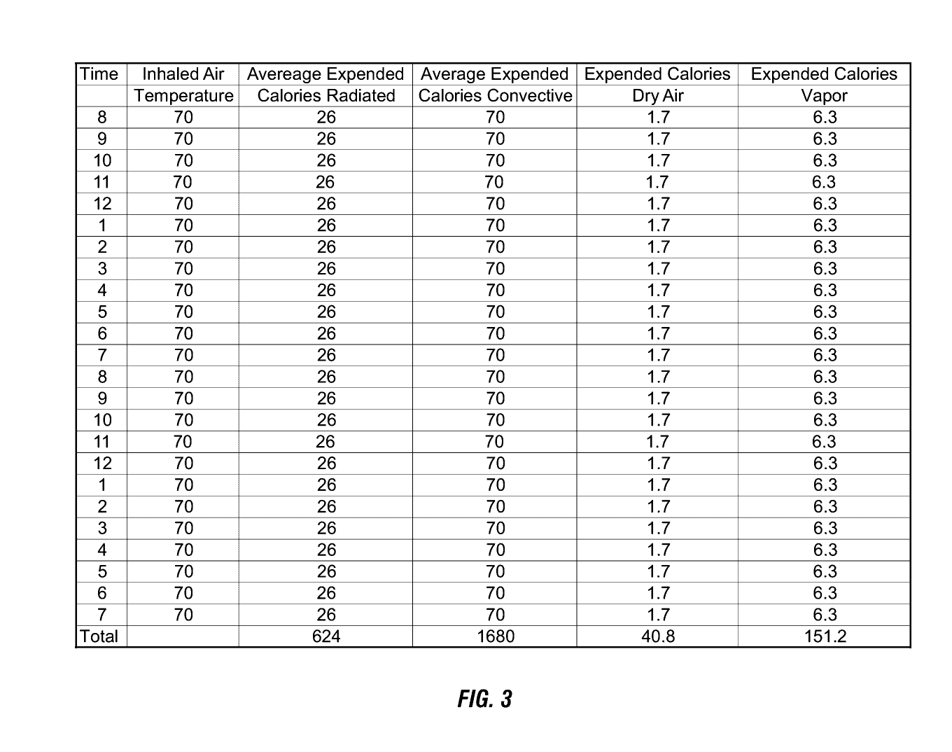

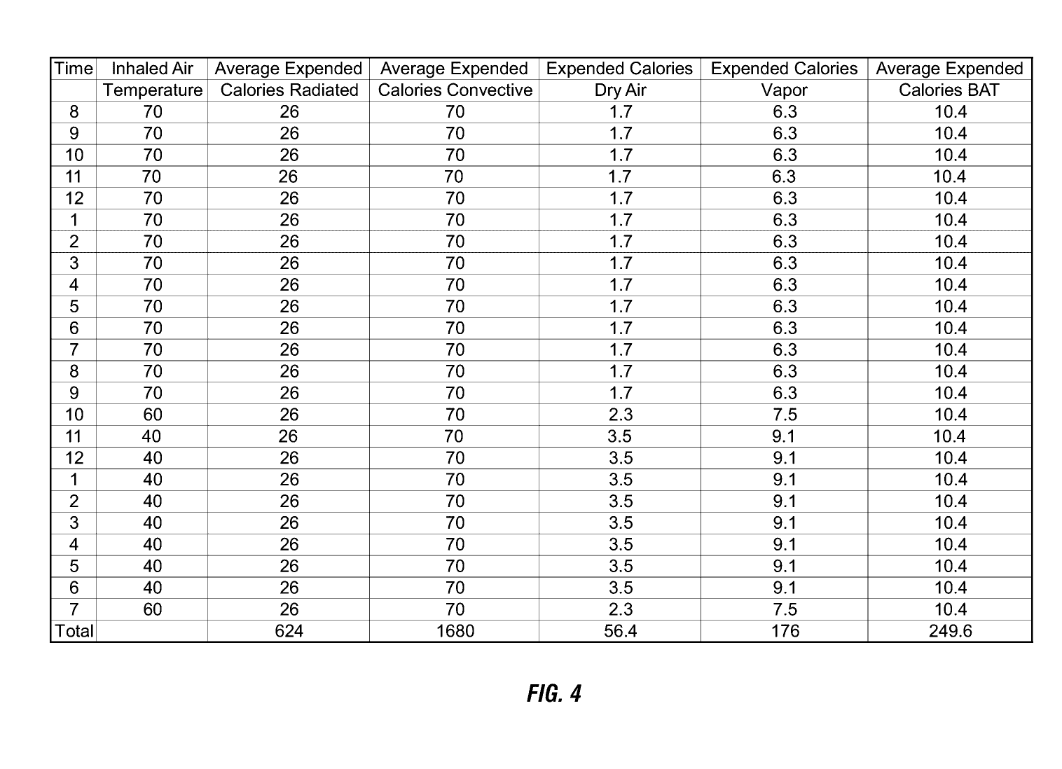

Tables 3 and 4 compare estimated average daily caloric expenditures for a person using a conventional CPAP machine and a person using the cold air CPAP machine of the present invention. In Table 1, it is presumed that the room temperature where the person sleeps is 70° F. In comparison, Table 2 shows the reduced temperature air at a 40° F. temperature for approximately six hours using the cold air CPAP machine of the present invention. Both tables are FIGS. 3 and 4 show radiated and convective calories expenditures, which do not change with the present invention, since these calories expended rely on surface area of the body and are not affected by breathing cooler air. In comparison, the dry air and vapor calories expended increase when the cold air is breathed, as seen in columns 5 and 6 of the tables. The increased dry air and vapor caloric expenditure are realized beginning on the first day of cold air therapy. The last column of FIG. 4 shows additional calories expended due to the increased production and activity of BAT. Since BAT develops over time using the cold air CPAP machine of the present invention, the BAT calories expended are estimated after six weeks of cold air therapy. The BAT calories expended is a conservative estimate, and may be approximately twice as high at 500 calories per day after six weeks of cold air breathing.

The increase in caloric expenditures from dry air, vapor and BAT is additive, such that the total estimated increased daily expenditure is 290 kilo calories, or approximately 2030 kilo calories extra per week, based upon the estimates of the table of FIG. 4. This conservative estimate is approximately equal to walking three miles a day every day, since one mile burns approximately 100 calories. Since white fat equals 4,000 calories per pound, the present invention may induce a loss of at least one pound of body fat every two weeks, in addition to weight loss derived from dietary restriction and exercise. Such weight loss will likely produce other health benefits from obesity related conditions, such as a decrease in insulin resistance.

From the foregoing, it can be seen that the present invention provides a practical and comfortable method and means for providing cool air for breathing during relative long duration while a person is asleep, so as to facilitate weight loss and blood sugar manipulation.

What is claimed is:

1. A method of increasing brown adipose tissue development in a person during sleep, comprising:
breathing air at a temperature no more than 55° F. through a CPAP mask worn by the person while asleep.

2. The method of claim 1 further comprising stepping down the air temperature over a period of time after the CPAP mask is positioned on the person.

3. The method of claim 2 wherein the air temperature drops to at least 55° F. after the person enters a first REM sleep cycle.

4. The method of claim 2 further comprising stepping up the air temperature to at least 60° F. before the person awakens.

5. The method of claim 1 further comprising monitoring the person's body temperature while asleep.

6. The method of claim 5 further comprising maintaining the body temperature above 94.1° F.

7. The method of claim 1 further comprising maintaining ambient air temperature above 60° F.

8. The method of claim 1 wherein inhalant air end product is maintained within a 10° F. range from the chiller.

9. The method of claim 1 wherein the breathing air temperature is in the range of 40°-55° F.

10. The method of claim 1 further comprising humidifying the air to at least 50% relative humidity before breathing.

11. A system for delivering cold air to a sleeping person, comprising:
a CPAP machine;
a mask connected to the CPAP machine;
an air chiller connected to the CPAP machine to reduce temperature of air to at least 55° F. for breathing by a person wearing the mask.

12. The system of claim 11 further comprising a humidifier between the air chiller and the mask to increase humidity of the air prior to breathing.

13. The system of claim 11 further comprising a sensor to sense the core body temperature of the person during sleep.

14. The system of claim 11 further comprising a processor connected to the air chiller to gradually reduce the air temperature from ambient to at least 55° F. after the person enters a first REM sleep cycle.

15. The system of claim 14 wherein the processor raises the air temperature to ambient before the person exits a last REM sleep cycle.

16. A method of increasing energy expenditure by a person, comprising:
placing a breathing mask on the person to deliver air to the person for breathing; and
cooling air delivered by the mask to the person for breathing to at least 55° F. while the person sleeps.

17. The method of claim 16 wherein the mask is connected to a CPAP machine.

18. The method of claim 16 further comprising raising humidity of the air delivered by the mask.

19. The method of claim 16 further comprising monitoring the person's core body temperature.

20. The method of claim 1 further comprising lowering the air temperature until the person's core body temperature is approximately 95° F.

21. The method of claim 16 wherein the air temperature is lowered after the person enters a first REM sleep cycle.

22. The method of claim 17 wherein the air temperature is raised to ambient before the person exits a last REM sleep cycle.
